(19) United States
(12) Patent Application Publication
(10) Pub. No.: US 2002/0169443 A1
(43) Pub. Date: Nov. 14, 2002

(54) Magnetized accupressure apparatus

(76) Inventor: Jung Pil Kim, Torrance, CA (US)

Correspondence Address: JONATHAN Y. KANG, ESQ., LEE & HONG P.C., 11th Floor, 221 N. Figueroa Street, Los Angeles, CA, 90012-2601, US

(21) Appl. No.: 09/853,361

(22) Filed: May 10, 2001

(51) Int. Cl. A61N 2/00

(52) U.S. Cl. 606/11; 600/9; 606/15; 714/786

(57) ABSTRACT

An apparatus for applying magnetized accupressure therapy to an area of the human body uses a parabolic reflector to converge the magnetic energy onto a focal point of the parabolic reflector. The apparatus comprises a body having a proximal end and a distal end. The body is magnetized to provide magnetic therapy. The apparatus also comprises a parabolic reflector attached to the proximal end of the body and a pin extending from the proximal end of the body for placing on a skin surface. The reflector has a focal point and focal length to converge magnetic energy thereto.

### BACKGROUND OF THE INVENTION

1. Field of the Invention

The present invention relates to a handheld accupressure apparatus, and more particularly, to a magnetic therapy accupressure apparatus to ease pain and facilitate healing.

2. Discussion of the Related Art

Magnetic and accupressure therapy is well known and widely used in the treatment of various ailments. Many products are available for practicing magnetic therapy. Generally, these products are attachable or wearable items which contain pockets holding permanent magnets so that magnetic fields may be applied to parts of the body.

It is believed that the magnetic fields act upon hemoglobin in the blood and enhance the ability of the blood to carry oxygen and nutrients to various parts of the body. In particular, it is known that the blood transports essential substances, such as oxygen and enzymes to the cells. For example, oxygen is carried by hemoglobin in the red blood cells, which contains iron, and carbon dioxide is carried in the blood plasma as bicarbonate. It is believed that after passing through the magnetic field produced by the present invention, certain important components of the blood are ionized by magnetic induction and the ability of the blood to carry components, such as oxygen, nutrients and waste products is significantly improved. This is equivalent to improving the circulation, and results in accelerated healing, pain reduction, and a general improvement in the condition of the body tissues.

Magnetic therapy is sometimes performed by physiotherapists using a pulsed magnetic field to alleviate certain diseases and generally improve the health of the recipient. However, the equipment used is large and extremely expensive and would not be practical for use on a personal basis.

### SUMMARY OF THE INVENTION

Accordingly, the present invention is directed to a magnetized accupressure apparatus that substantially obviates one or more of the problems due to limitations and disadvantages of the related art.

It is therefore an object of the present invention to provide an apparatus which is adapted for the application of magnetic therapy.

It is another object of the present invention to provide a device that can focus the magnetic field generated from such apparatus onto a small region.

It is another object of the present invention to provide an apparatus which is easy to manufacture.

It is still another object of the present invention to provide an apparatus which may be manufactured in a variety of shapes and sizes for application to different parts of the body.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, a magnetic therapy apparatus comprises a body having a proximal end and a distal end. The body is magnetized to provide magnetic therapy. The apparatus also comprises a parabolic reflector attached to the proximal end of the body and a pin extending from the proximal end of the body for placing on a skin surface. The reflector has a focal point and length to converge magnetic energy thereto.

According to one aspect of the present invention, the pin is extending from a boresight of the parabolic reflector. Preferably, the pin has a length substantially the same as the focal length of the parabolic reflector. In particular, the pin and the focal length are approximately 0.5 inches long.

According to another aspect of the present invention, the proximal end of the body is magnetized to have North pole and the distal end of the body has South pole.

According to another embodiment of the present invention, an apparatus for applying accupressure therapy to a subject comprises a U-shaped body having first and second proximal ends, wherein the body is magnetized; a first reflector attached to the first proximal end of the body, wherein the first reflector has a first focal point to converge magnetic energy thereto; a second reflector attached to the second proximal end of the body, wherein the second reflector has a second focal point to converge magnetic energy thereto; a first pin extending from the first proximal end of the body; and a second pin extending from the second proximal end of the body.

According to one aspect of the present invention, the first and the second reflectors are parabolic reflectors, each parabolic reflector having a focal length.

According to one aspect of the present invention, a length of each one of the first and the second pins is substantially equal to the focal length.

It is to be understood that both the foregoing general description and the following detailed description are exemplary and explanatory and are intended to provide a further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and, together with the description, serve to explain the principles of the invention.

FIG. 1 illustrates a perspective view of an accupressure apparatus according to a preferred embodiment of the present invention;

FIG. 2 illustrates a cross-sectional view of FIG. 1;

FIG. 3 illustrates the use of the present invention on user's skin; and

FIG. 4 illustrates a perspective view of a second embodiment of the accupressure apparatus.

### DETAILED DESCRIPTION OF THE PREFERRED EMBODIMENTS

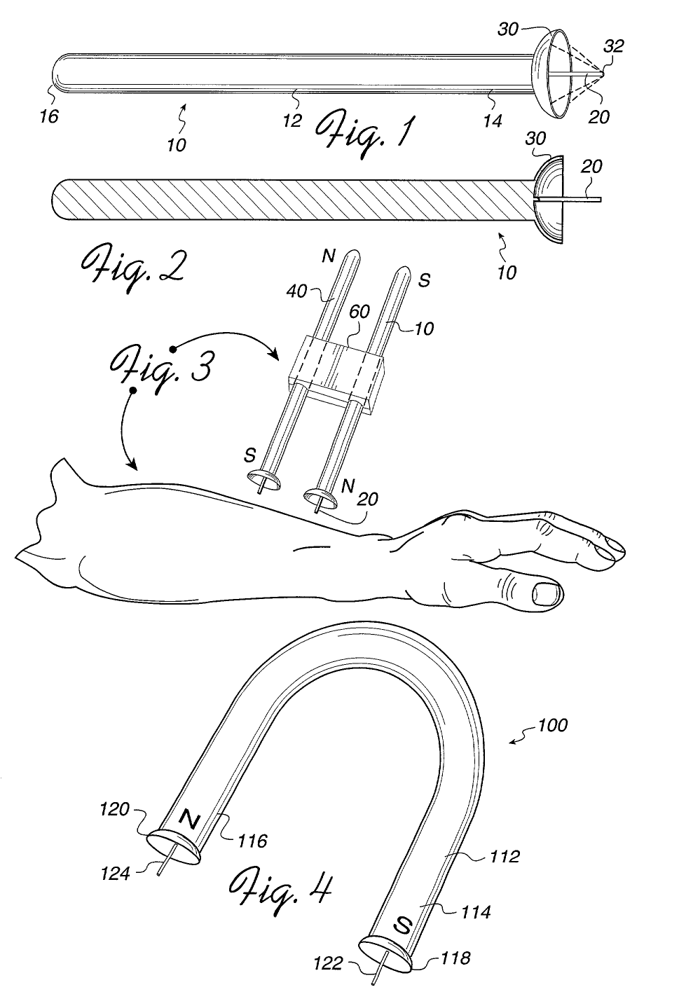

With reference to the drawings, and in particular to FIGS. 1-4 thereof, a magnetized accupressure apparatus embodying the principles and concepts of the present invention will be described.

The accupressure apparatus 10 according to the preferred embodiment of the present invention enhances healing and has therapeutic effect by using magnetized accupressure pins and parabolic reflectors on one end of a magnetized rod. The therapeutic effects include treating sprains, strains and soothing sore and aching muscles.

FIG. 1 illustrates a perspective view of the preferred embodiment of the accupressure apparatus 10. The accupressure apparatus 10 includes an elongated body 12 in the form of a rod having proximal end 14 and distal end 16. The elongated body 12 is preferably magnetized so that each end of the body 12 is polarized. For example, in one embodiment, the body 12 is magnetized such that the proximal end 14 has a polarity of south pole and the distal end 16 has a polarity of north pole. In another embodiment, the polarity may be switched so that the proximal end 14 and the distal end 16 have north and south poles, respectively.

In the preferred embodiment, there is provided a reflector 30 at the proximal end 14. The reflector 30 has a concaved surface adapted to converge the energy toward a small region or a focal point. Preferably, the reflector is a parabolic reflector 30 and is securely attached to the proximal end 14. The parabolic reflector 30 has a focal point 32 which is located at a focal length and is used to focus magnetic field energy at the focal point 32.

Parabolic dishes are typically used to focus radio signals, solar rays, etc. on a focal point. Similarly, the parabolic reflector 30 uses the same characteristic of the parabolic shape to focus magnetic field energy at a small region. By using the focusing characteristics of the parabolic reflector 30, the focal point 32 of the accupressure apparatus 10 has magnetic field that approximately twice the strength compared to a magnetized rod without the parabolic reflector 30. Preferably, the parabolic reflector 30 may be made of the same material as the body 12 or other suitable materials known to one of ordinary skill in the art.

According to the preferred embodiment, the distal end 16 of the body 12 has a rounded shape for use as accupressure and massage therapy. The user may use the distal end 16 to apply accupressure against any skin surface.

Referring to FIGS. 1 to 3, longitudinally extending from the proximal end 14 of the body 12 is an elongated pin 20. The pin 20 is preferably attached at the boresight of the parabolic reflector 30 and forms an integral structure with the body 12. A tip of the pin 20 is preferably blunt and not sharp to prevent skin penetration when the accupressure apparatus 10 is applied again the skin surface. The length of the pin 20 is substantially the same as the focal length of the parabolic reflector 30. In the preferred embodiment, the length of the pin 20 is about 0.5 inches. Preferably, the thickness of the pin is about 1/128 inches. However, other pin thickness may be chosen without deviating from the gist of the present invention.

According to the present invention, the body 12 of the accupressure apparatus 10 has a length of about 6 to 7 inches and a diameter of about 0.2 to 0.3 inches. The body 12 is preferably made of magnetic materials or materials that can be magnetized. The parabolic reflector 30 has a reflector diameter of about 0.5 inches.

Preferably, alnico magnets are used which are primarily made from aluminum, nickel, cobalt, copper, iron and sometimes titanium. Alnico is melted and poured into a mold. Once solidified, the material is heat treated and cooled in a magnetic field. When treated in the presence of a magnetic field, the magnet is called anisotropic. Such process provides the material to take on maximum magnetization and allows a higher gauss level. Alternatively, in lieu of treating the material in the presence of a magnetic field, the material may be machined to the correct shape and tolerances and then magnetized.

Although the present invention may be singularly used on a skin surface, a preferable therapeutic treatment is by using a pair of the accupressure apparatus as shown in FIG. 3. Referring to FIG. 3, there is provides a holder 60 adapted to receive two accupressure apparatus 10 and 40 separated by a predeterminal distance, for example approximately 1 to 2 inches. A first accupressure apparatus 10 is magnetically polarized to have a south pole in the proximal end and a north pole in the distal end. A second accupressure apparatus 40 is magnetically polarized to have a north pole in the proximal end and a south pole in the distal end. This arrangement produces a pair of different polarization, namely north and south poles, adjacent to each other. Thus, as blood flows through the veins of the wrist in one direction, the blood passes through north and south poles of the magnetic field.

Because oxygen is carried by hemoglobin in the red blood cells, which contains iron, and carbon dioxide is carried in the blood plasma as bicarbonate. It is believed that after passing through the magnetic field produced by the present invention, certain important components of the blood are ionized by magnetic induction and the ability of the blood to carry components, such as oxygen, nutrients and waste products is significantly improved.

In this regard, using the parabolic reflector 30 with the magnetized body 12 to focus the magnetic field energy reduces recovery time of certain ailments by a factor of at least one-half or more. In chronic conditions, such as arthritis, degenerative joint conditions and ulcers, the accupressure apparatus 10 having the parabolic reflector 30 has shown dramatic results in aiding the reduction or reversal of such conditions. Moreover, because the pin 20 does not actually penetrate the skin surface, there is less or no pain and the excretion of blood is not involved.

Like a satellite dish, the parabolic reflector 30 focuses the magnetic field towards the tip of the pin 20, which is also the focal point 32 of the reflector 30. This structure allows maximum force of the magnetic field to be focused onto the focal point 32 thus into the treatment area.

The accupressure apparatus 10 according to the present invention may be assembled in different sizes without deviating from the gist of the present invention. As bigger body 12 is used, the magnetic force may be increased accordingly.

FIG. 4 illustrates a perspective view of a second embodiment of the accupressure apparatus. Referring to FIG. 4, the accupressure apparatus 100 has a body 112 that has a shape of a typical horseshoe magnet. In particular, the body 112 is of a U-shaped rod and has two proximal ends 114 and 116. A first proximal end 114 is polarized as a south pole S and a second proximal end 116 is polarized as a north pole N.

According to the preferred embodiment, there are provided parabolic reflectors 118 and 120 at the proximal ends 114 and 116 of the body 112. Each parabolic reflector 118 and 120 is identical to one described with respect to the first embodiment of the present invention, and thus its description will not be repeated for the sake of brevity.

At each end of the body 112, there is provided a pin 122, 124. In particular, at the first proximal end 114, there is a first pin 122 extending from the boresight of the first parabolic reflector 118. Similarly, at the second proximal end 116, there is a second pin 124 extending from the boresight of the second parabolic reflector 120. Each pin is approximately 0.5 inches long to substantially match the focal length of the parabolic reflectors 118 and 120.

As described above, the parabolic reflectors 118 and 120 are used to focus magnetic field energy at each respective focal point. By matching the length of the pins 122 and 124 with the focal points of the parabolic reflectors, increased magnetic energy is applied into the user's skin.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. An apparatus for applying accupressure therapy to a subject, comprising:
a body having a proximal end and a distal end, wherein the body is magnetized;
a reflector attached to the proximal end of the body, wherein the reflector has a focal point to converge magnetic energy thereto; and
a pin extending from the proximal end of the body for placing on the subject.

2. The apparatus of claim 1, wherein the reflector is a parabolic reflector having a focal length.

3. The apparatus of claim 2, wherein the pin is extending from a boresight of the parabolic reflector.

4. The apparatus of claim 2, wherein the pin has a length substantially the same as the focal length of the parabolic reflector.

5. The apparatus of claim 1, wherein the proximal end of the body is magnetized to have North pole and the distal end of the body has South pole.

6. The apparatus of claim 1, wherein the proximal end of the body is magnetized to have South pole and the distal end of the body has North pole.

7. The apparatus of claim 4, wherein the pin is approximately 0.5 inches long.

8. The apparatus of claim 1, wherein the body is of a U-shaped rod.

9. The apparatus of claim 8, wherein each end of the U-shaped rod has a parabolic reflector having a focal point to converge the magnetic energy thereto.

10. The apparatus of claim 9, wherein each end of the U-shaped rod has a pin extending therefrom, wherein the pin has a length substantially equal to a focal length of the parabolic reflector.

11. An apparatus for applying accupressure therapy to a subject, comprising:
a body having a proximal end and a distal end, wherein the body is magnetized;
reflector means for focusing magnetic energy from the body onto a focal point, the reflector means being attached to the proximal end of the body; and
probing means for applying pressure on the subject, the probing means protruding from the reflector means.

12. The apparatus of claim 11, wherein the reflector means is a parabolic reflector having a focal length.

13. The apparatus of claim 11, wherein the probing means is extending from a boresight of the reflector means.

14. The apparatus of claim 11, wherein the reflector means has a focal length and the probing means has a length substantially the same as the focal length.

15. An apparatus for applying accupressure therapy to a subject, comprising:
a U-shaped body having first and second proximal ends, wherein the body is magnetized;
a first reflector attached to the first proximal end of the body, wherein the first reflector has a first focal point to converge magnetic energy thereto;
a second reflector attached to the second proximal end of the body, wherein the second reflector has a second focal point to converge magnetic energy thereto;
a first pin extending from the first proximal end of the body; and
a second pin extending from the second proximal end of the body.

16. The apparatus of claim 15, wherein the first and the second reflectors are parabolic reflectors, each parabolic reflector having a focal length.

17. The apparatus of claim 16, wherein a length of each one of the first and the second pins is substantially equal to the focal length.
